(19) United States
(12) Patent Application Publication
(10) Pub. No.: US 2006/0227979 A1
(43) Pub. Date: Oct. 12, 2006

(54) Contact type electronic stethoscope with a noise interference resisting function for auscultation

(75) Inventor: Sue-Fen Chen, Taipei (TW)

Correspondence Address: BACON & THOMAS, PLLC, 625 SLATERS LANE, FOURTH FLOOR, ALEXANDRIA, VA, 22314, US

(73) Assignee: HEALTH & LIFE CO., LTD, CHUNG HO CITY (TW)

(21) Appl. No.: 11/100,438

(22) Filed: Apr. 7, 2005

(51) Int. Cl. A61B 7/04

(52) U.S. Cl. 381/67

(57) ABSTRACT

The present invention describes a contact type electronic stethoscope with a noise interference resisting function for auscultation, and the stethoscope includes a chest piece for contacting a patient's body, a contact type microphone installed in the chest piece for receiving sounds of patient's organs, and an elastic member disposed on an external side of the contact type microphone, so that harsh sounds and irrelevant noises produced by rubbing the surface of clothes with the patient's body can be avoided during an auscultation, if the piece chest is pressed and contacted with the patient's body without reaching a predetermined pressure and maintains a predetermined distance from the patient's body.

### BACKGROUND OF THE INVENTION

1. Field of the Invention

The present invention generally relates to stethoscopes, and more particularly relates to a contact type electronic stethoscope with a noise interference resisting function for auscultation, which is used for receiving the physiological sound signals produced by human organs.

2. Description of the Related Art

In general, traditional stethoscopes are divided into cable stethoscopes and wireless electronic stethoscopes, and the operational principle of receiving digital signals by a contact type wireless electronic stethoscope includes a chest piece in contact with a patient's body, a contact type microphone installed in the chest piece, a circuit board having an audio frequency logic circuit, an LCD output circuit, an output transmission circuit, a press key input logic circuit and a function select circuit. If the chest piece is placed in contact with a patient's body, the contact type microphone will receive analog sounds produced by the patient's organs, and the audio frequency is converted into a digital signal of the logic circuit. After the signal is processed by a CPU, the LCD output circuit displays related information on an LCD screen, and then the output transmission circuit transmits an audio signal from a signal transmitter, and thus the sound can be heard from an earpiece.

However, the aforementioned prior art contact electronic stethoscope has a drawback of producing harsh sounds by rubbing the chest piece with the patient's body or the surface of clothes and being misled by irrelevant noises during an auscultation, and thus affecting the hearing of medical professionals. Medical professionals have to spend more efforts to listen carefully to the related weak sound signals and tolerate the noises and interferences of frictions. In addition, a wireless digital electronic stethoscope will pick up noises irrelevant to patient sounds and cause a transmission of wrong signals.

### SUMMARY OF THE INVENTION

The present invention is provided to overcome the foregoing shortcomings of the prior art contact type stethoscope, and thus it is a primary objective of the present invention to provide a contact type electronic stethoscope with a noise interference resisting function for auscultation, so as to prevent interferences and noises produced by rubbing the chest piece with the patient's body, and allow medical professions to determine the time for listening to and receiving signals by directly pressing a press button or applying a force onto the chest piece.

Another objective of the present invention is to provide a contact type electronic stethoscope with a noise interference resisting function for auscultation, such that an audio data is processed by a CPU and sent to a receiving end of a wireless earphone, a computer, or a PDA through a wireless transmission.

To achieve the foregoing objectives, the contact type electronic stethoscope with a noise interference resisting function for auscultation has a chest piece, and the chest piece includes a circuit board, an integrated circuit chip and a contact type microphone installed in the chest piece. The contact type microphones receives the audio frequencies of various sounds produced by a patient's organs such as respiratory tract, lungs, heart, intestines, stomach and digestive tract. With other receivers, the audio frequency is transmitted to an earpiece through a wireless transmission, so that medical professions can listen directly to the receiver or observe the data displayed on an LCD screen which is installed on an external side of the stethoscope to assist medical professional to obtain an accurate diagnosis during an auscultation.

### BRIEF DESCRIPTION OF THE DRAWINGS

FIG. 1 is schematic view of a prior art contact type electronic stethoscope;

FIG. 2 is a schematic view of a preferred embodiment of the invention;

FIGS. 3 and 4 are schematic views of the operations of the invention;

FIG. 5 is a schematic view of another preferred embodiment of the invention;

FIG. 6 is a perspective view of a further preferred embodiment of the invention; and

FIG. 7 is a schematic view of another further preferred embodiment of the invention.

### DETAILED DESCRIPTION OF THE PREFERRED EMBODIMENTS

To make it easier for our examiner to understand the present invention, the following detailed description with reference to the accompanying drawings of an embodiment are given for example, but such preferred embodiment is not intended to limit the scope of the present invention.

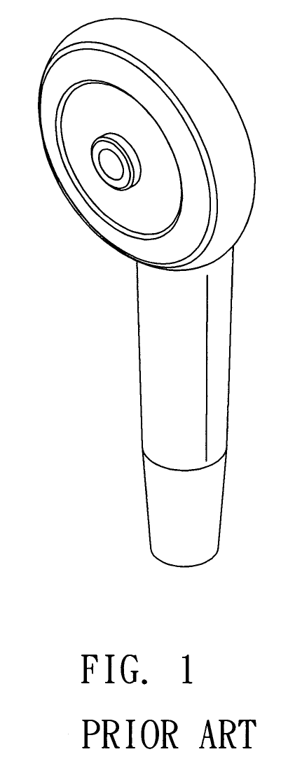

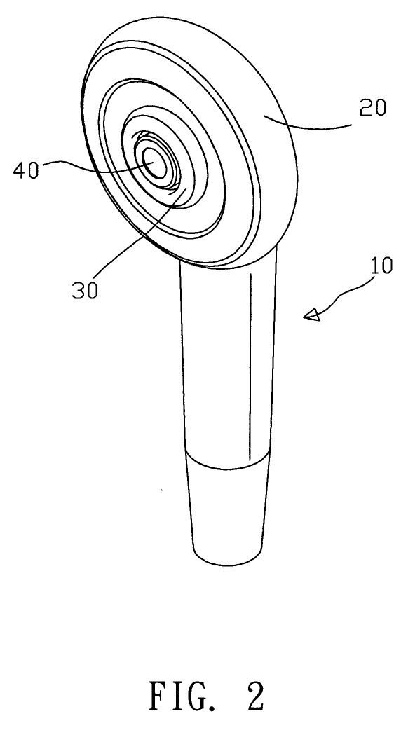

Referring to FIG. 1, a schematic view of a prior art contact type electronic stethoscope is shown. Referring to FIG. 2, a contact type electronic stethoscope with a noise interference resisting function for auscultation according to a first preferred embodiment of the present invention is illustrated. The contact type electronic stethoscope 10 comprises a chest piece 20 disposed at an end proximate to a patient's body and having a contact type microphone 40 installed in the chest piece 20 and an elastic member 30 disposed at an external side of the contact type microphone 40; a circuit board (not shown in the figure) having a CPU (not shown in the figure), a logic circuit (not shown in the figure) and a signal generator (not shown in the figure); and a contact type microphone 40, installed at the central position of the chest piece 20 of the contact type electronic stethoscope 10 of the invention, such that the contact microphone is electrically coupled to the circuit board. If the elastic member 30 is pressed by appropriate pressure to produce a deformation, the contact type microphone 40 will be able to contact a patient's body to receive physiological sounds including heart tones, lung sounds, internal organ sounds and the like produced by the patient's organs.

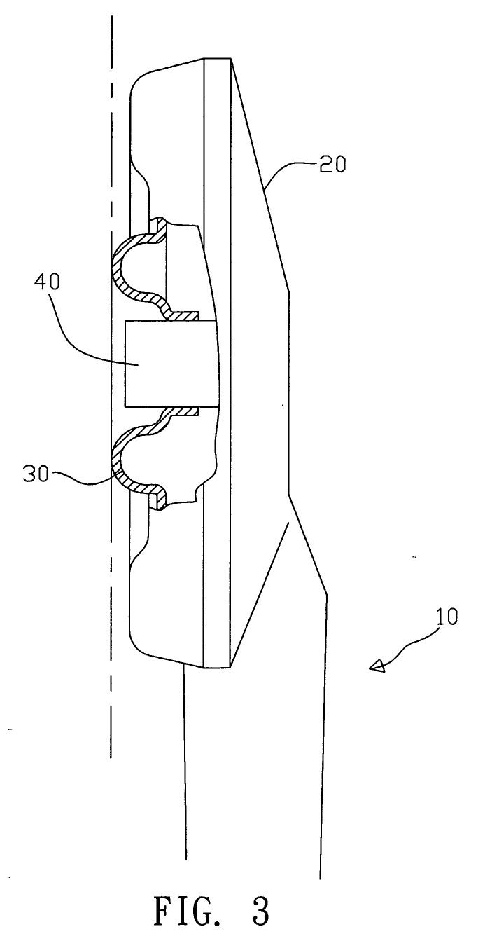

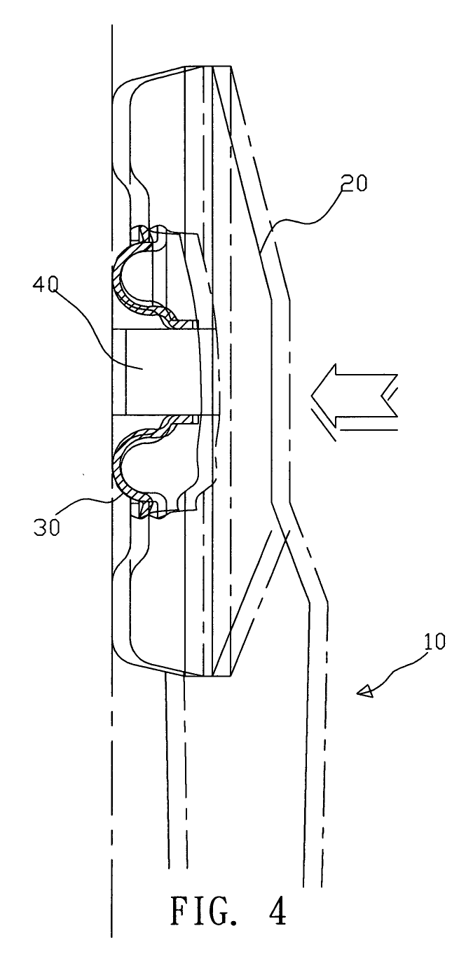

Referring to FIGS. 3 and 4, schematic views of the operations of the invention are shown. In FIG. 3, a medical professional needs not to apply a force on the contact type electronic stethoscope 10 if the chest piece 20 has not been placed onto a desired position of the patient's body yet. At this moment, the elastic member 30 is protruded to a certain height, so that the contact type microphone 40 is maintained at a certain distance from the patient's body. The noise produced by rubbing the contact microphone 40 with the patient's body or the surface of clothes will not be sent out.

Referring to FIG. 4 for a schematic view of a deformation of an elastic member 30 produced when a medical professional moves the chest piece 20 to a desired position of the patient's body and presses the external side of the chest piece 20, the contact type microphone 40 in the chest piece 20 is in contact with the patient's body due to the compressed and deformed elastic member 30, and thus can receive the physiological sounds produced by the patient's organs. Since the chest piece 20 comes with the elastic member 30, therefore the stethoscope 10 has a soundproof function and gives a more comfortable feeling to patients instead of a sharp and cold contact.

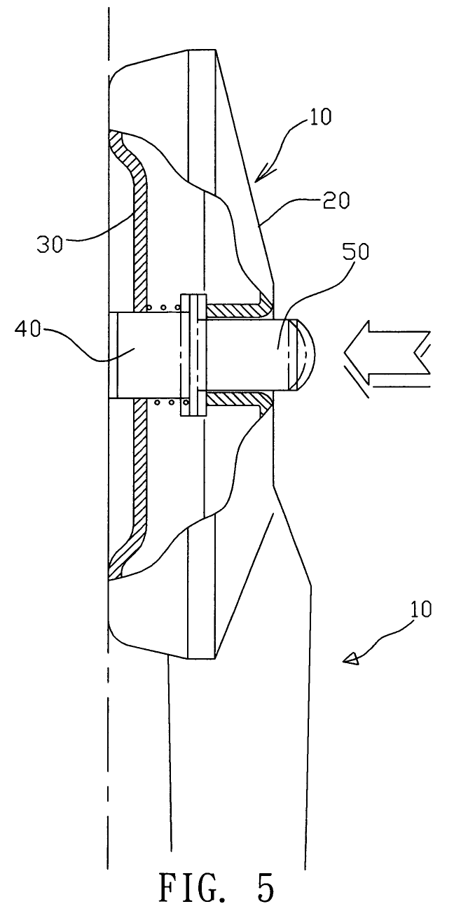

Referring to FIG. 5, another preferred embodiment of the invention is illustrated. Unlike the first preferred embodiment, the contact type microphone 40 of this embodiment is installed in an internal side of the chest piece 20, and an elastic member 30 is installed on an external side of the contact type microphone 40, so that the elastic member 30 can be compressed or released by pressing or releasing a press button 50 disposed on an external side of the chest piece 20, and the press button 50 controls the forward movement and the withdrawal of the contact type microphone 40. The elastic member 30 is normally kept at a released status. The contact type microphone 40 will be stored in the internal side of the chest piece 20 and will not be in contact with the patient's body, if the chest piece 20 is not placed at a desired position of the patient's body. Therefore, noises caused by moving the chest piece 20 will not be sent out. If the chest piece 20 is placed at a desired position of the patient's body for a medical professional to listen to the physiological sounds, the medical professional just needs to gently press the press button 50 to push the contact type microphone 40 to a predetermined position, and the contact type microphone 40 will contact the patient's body to receive the physiological sounds produced by the patient's organs. After the auscultation ends, the press button 50 is released to return the elastic member 30 to its released position and also return the contact type microphone 40 to its original position.

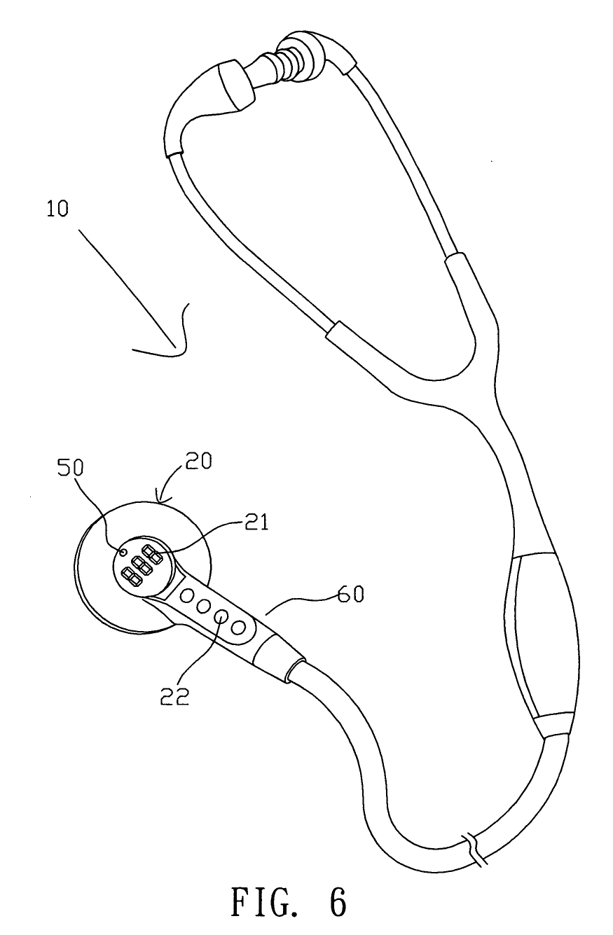

Referring to FIG. 6, a schematic view of another preferred embodiment of the present invention is shown. The chest piece 20 of the contact type electronic stethoscope 10 includes an LCD screen 21 on its external side. After the physiological sounds produced by the patient's organs are received by the contact type microphone 40 and digitized to produce digital signals, the digital signals are transmitted via a cable and displayed on the LCD display 21. A control switch 22 is installed at a handle section 60 of the contact type electronic stethoscope 10 for controlling the volume of output sounds.

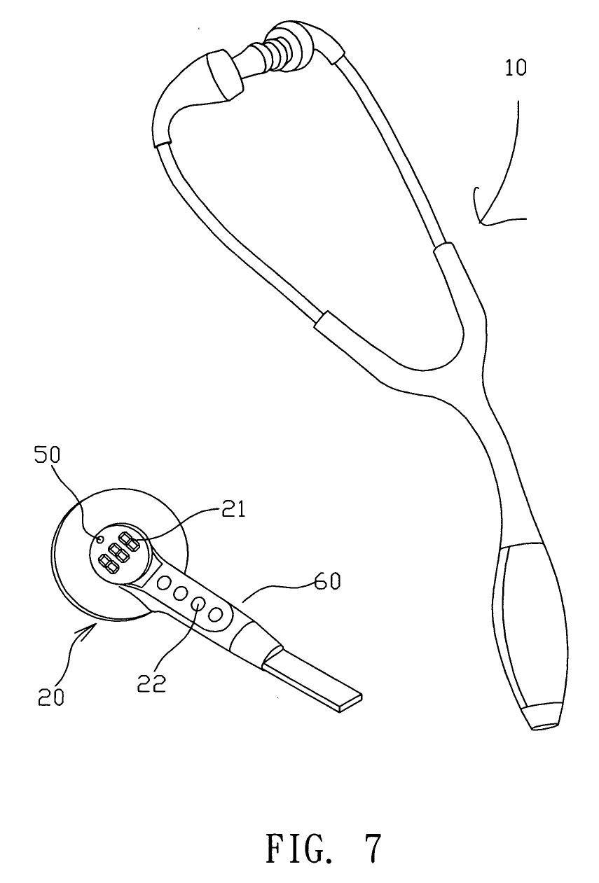

Referring to FIG. 7, a schematic view of another further preferred embodiment of the present invention is shown. The chest piece 20 of the contact type electronic stethoscope 10 includes an LCD screen 21 on its external side. After the physiological sounds produced by the patient's organs are received by the contact type microphone 40 and digitized to produce digital signals, the digital signals are transmitted via a wireless transmission and displayed on the LCD display 21. A control switch 22 is installed at a handle section 60 of the contact type electronic stethoscope 10 for controlling the volume of output sounds.

In summation of the description above, the present invention complies with the patent application requirements. The description and its accompanied drawings are used for describing preferred embodiments of the present invention, and it is to be understood that the invention is not limited thereto. To the contrary, it is intended to cover various modifications and similar arrangements and procedures, and the scope of the appended claims therefore should be accorded the broadest interpretation so as to encompass all such modifications and similar arrangements and procedures.

What is claimed is:

1. A contact type electronic stethoscope with a noise interference resisting function for auscultation, comprising:
a chest piece, disposed on said stethoscope at a position in contact with a patent's body;
a contact type microphone, installed in the middle of said chest piece;
an elastic member, installed at an external side of said contact type microphone; and
a circuit board, electrically coupled with said contact type microphone;
thereby when said elastic member is pressed by an appropriate pressure to produce a deformation, said contact type microphone is in contact with said patent's body to receive a physiological sound produced by a patient's organ.

2. The contact type electronic stethoscope with a noise interference resisting function for auscultation of claim 1, wherein said contact type microphone is pushed out or withdrawn by controlling a press button.

3. The contact type electronic stethoscope with a noise interference resisting function for auscultation of claim 1, wherein said stethoscope comprises a display disposed on an external side said stethoscope for displaying a detected signal.

4. The contact type electronic stethoscope with a noise interference resisting function for auscultation of claim 2, wherein said stethoscope comprises a display disposed on an external side said stethoscope for displaying a detected signal.

5. The contact type electronic stethoscope with a noise interference resisting function for auscultation of claim 1, wherein said circuit board comprise a transmission circuit for a wireless transmission.

6. The contact type electronic stethoscope with a noise interference resisting function for auscultation of claim 2, wherein said circuit board comprise a transmission circuit for a wireless transmission.

7. The contact type electronic stethoscope with a noise interference resisting function for auscultation of claim 1, wherein said circuit board comprises a control circuit for adjusting the volume of an output sound

8. The contact type electronic stethoscope with a noise interference resisting function for auscultation of claim 2, wherein said circuit board comprises a control circuit for adjusting the volume of an output sound.
